(19) United States
(12) Patent Application Publication
(10) Pub. No.: US 2005/0019296 A1
(43) Pub. Date: Jan. 27, 2005

(54) Preparation of saccharide esters

(75) Inventors: Franz-Xaver Scherl, Burgkirchen (DE); Peter Klug, Grossostheim (DE); Claudia Potyka, Kastl (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 10/859,928

(22) Filed: Jun. 3, 2004

(30) Foreign Application Priority Data
Jun. 4, 2003 (DE) 10325200.2

(51) Int. Cl. A61K 7/32; C07H 13/02

(52) U.S. Cl. 424/70.13; 536/119

(57) ABSTRACT

A process for the preparation of saccharide esters by reacting saccharides with activated carboxylic acids in a dipolar-aprotic solvent and in the absence of a basic amine compound is described.

The present invention relates to a process for the preparation of saccharide esters in aprotic solvents without the addition of an amine base.

Consumer wishes and rheology of cosmetic products are closely related. Thus, for example, the visual appearance of a cream or lotion is influenced by the viscosity. The sensory properties, such as consistency or spreadability, determine the individual profile of a cosmetic product. The effectiveness of active substances (e.g. deodorants, sunscreen filters) and also the storage stability of the formulation is also closely related to the rheological properties of the product. In the cosmetics sector, the thickeners and gel formers therefore play a major role.

A number of patent specifications describe the use of polyol esters as thickeners.

DE 37 26 015 describes the reaction products of polyalcohols with fatty acids, for example pentaerythritol fatty acid esters, and their thickening action. Highly thixotropic agents for thickening oil phases can be prepared using dextrin esters, the preparation of which is described in U.S. Pat. No. 5,840,883. The reaction of dextrin with a fatty acid halide or anhydride takes place in dimethylformamide or formamide, acetamide compounds, ketone compounds, aromatics, for example benzene, toluene, xylene, dioxane, as solvent or dispersant in the presence of a tertiary amine, for example pyridine, triethylamine or picoline. Tertiary amines are toxic, impair the odor of the composition and require high expenditure with regard to purification of the dextrin esters and work-up of the waste substances produced in the preparation. WO 00/61079 and U.S. No. 2002/0072506 describe the preparation of cellobiose esters by reacting cellobiose and acid anhydride without a diluent, but the yields obtained are unsatisfactory.

The object was therefore to find a novel process for the preparation of saccharide esters which produces high product yields and good grades.

Surprisingly, saccharide esters can be obtained in very good yields if saccharides are reacted with activated carboxylic acids in the absence of an amine base, such as, for example, pyridine, and the esterification of the saccharides takes place in a dipolar-aprotic solvent.

The invention provides a process for the preparation of saccharide esters by reacting saccharides with activated carboxylic acids in a dipolar-aprotic solvent and in the absence of a basic amine compound.

As well as the high yields which can be achieved by the process according to the invention, it is likewise advantageous that solvent mixtures of dipolar-aprotic solvents and substances such as, for example, alcohols or water can be recycled easily, meaning that only a small amount of solvent is required for the preparation of the esters. A further advantage of the process according to the invention is that it is possible to dispense with the use of amines, which are problematic in terms of odor and toxicology.

Dipolar-aprotic solvents which can be used in the process according to the invention are, for example, lactams, formamides, such as, for example, dimethylformamide, and sulfoxides. Preference is given to using lactams as solvents, particularly preferably 2-methylpyrrolidone (NMP).

The process according to the invention for the esterification of saccharides includes the esterification of monosaccharides and of oligo- and polysaccharides, such as, for example, of disaccharides.

Preferably, oligo- and polysaccharides including disaccharides are esterified by the process according to the invention.

Among the disaccharides, preference is given to using cane sugar, milk sugar, trehalose, lactose, maltose, gentiobiose, melibiose and cellobiose in the process according to the invention.

As well as the disaccharides, among the oligo- and polysaccharides, preference is given to using cellotriose, cellotetrose, raffinose, acarbose, but also starch and constituents thereof, amylose, amylopectin, and dextrins, dextrans, xanthans or else cellulose in the process according to the invention.

In a preferred embodiment of the invention, saccharides chosen from dextrin and cellobiose are used in the process, i.e. compounds chosen from dextrin esters and cellobiose esters are prepared. The dextrins preferably have a degree of polymerization of from 3 to 200, particularly preferably from 5 to 100 and especially preferably from 10 to 50.

As activated carboxylic acids, preference is given to using substances chosen from carbonyl chlorides and carboxylic anhydrides in the process according to the invention.

For the esterification of the mono-, oligo- and polysaccharides, a preferred embodiment of the invention uses activated saturated or unsaturated aliphatic, cyclic-aliphatic or aromatic carboxylic acids having 2 to 30 carbon atoms.

The acids on which the activated carboxylic acids are based are, for example, caprylic acid, capric acid, pelargonic acid, lauryl acid, lauric acid, myristic acid, myristyl acid, palmitic acid, palmityl acid, stearic acid, arachic acid, behenic acid, oleic acid, erucic acid, gadoleic acid, linoceryl acid, fish oil acid and soybean oil fatty acid, and derivatives thereof.

Further acids on which the activated carboxylic acids are based are, for example, short-chain acids, e.g. acetic acid, propionic acid and butyric acid, and derivatives thereof.

Further acids on which the activated carboxylic acids are based are, for example, branched, saturated carboxylic acids, e.g. isobutyric acid, isovaleric acid, 2-ethylbutyric acid, ethylmethylacetic acid, isoheptyl acid, 2-ethylhexyl acid, isononanoic acid, isodecanoic acid, isotridecanoic acid, isomyristyl acid, isopalmityl acid, isostearic acid, isoarachic acid and isohexacosanoic acid, and derivatives thereof.

Further acids on which the activated carboxylic acids are based are, for example, unsaturated carboxylic acids, e.g. cis-4-decenoic acid, 9-decenoic acid, cis-4-dodecenoic acid, cis-4-tetradecenoic acid, cis-5-tetradecenoic acid, cis-9-tetradecenoic acid, cis-6-hexadecenoic acid, cis-9-hexadecenoic acid, cis-9-octadecenoic acid, trans-9-octadecenoic acid, cis-11-octadecenoic acid, cis-11-eicosenoic acid, cis-17-hexacosenoic acid and cis-21 -tricontenoic acid, but also sorbic acid, linoleic acid, linolenic acid, gamma-linolenic acid, stearidonic acid, arachidonic acid and clupanodonic acid, and derivatives thereof.

Further acids on which the activated carboxylic acids are based are, for example, aromatic carboxylic acids, e.g. benzoic acid, anthranilic acid, salicylic acid, phthalic acid, terephthalic acid, isophthalic acid, and derivatives thereof, and araliphatic monocarboxylic acids, e.g. cinnamic acid and mandelic acid, and derivatives thereof.

Particularly preferred acids on which the activated carboxylic acids used in the process according to the invention are based are straight-chain or branched, saturated or unsaturated fatty acids having 8 to 22 carbon atoms.

In a further preferred embodiment of the invention, saccharides, preferably chosen from disaccharides, of these preference being given to cellobiose, and also oligo- and polysaccharides with degrees of polymerization of from 3 to 200, of these preference being given to dextrins, are esterified with a combination of two or more activated carboxylic acids.

Reaction products obtainable in this way are, for example, the following dextrin ester mixtures: dextrin ester of caprylic/isobutyric acid, dextrin ester of caprylic/2-ethylhexyl acid, dextrin ester of caprylic/isoarachic acid, dextrin ester of caprylic/linoleic acid, dextrin ester of caprylic/acetic acid, dextrin ester of caprylic/isopalmityl/butyric acid, dextrin ester of caprylic/palmityl/oleic acid, dextrin ester of caprylic/oleic/acetic acid, dextrin ester of lauryl/methylethyl acetic acid, dextrin ester of lauryl/2-ethylhexyl acid, dextrin ester of lauryl/capric acid, dextrin ester of lauryl/linolenic/propionic acid, dextrin ester of lauryl/behenic/isoheptanoic acid, dextrin ester of myristyl/isostearic acid, dextrin ester of myristyl/isohexanoic acid, dextrin ester of myristyl/arachidonic acid, dextrin ester of palmitic/2-ethylhexanoic acid, dextrin ester of palmitic/isostearic acid, dextrin ester of palmitic/oleic acid, dextrin ester of palmitic/isovaleric/isostearic acid, dextrin ester of palmitic/isononanoic/caproic acid, dextrin ester of palmitic/stearic/2-ethylhexanoic acid, dextrin ester of palmitic/stearic/caproic acid, dextrin ester of stearic/isopalmitic acid, dextrin ester of stearic/oleic acid, dextrin ester of behenic/2-ethylbutyric acid and dextrin ester of behenic/caproic/valeric acid.

All of the abovementioned dextrin ester mixtures can advantageously be prepared, for example, also on the basis of disaccharides, in particular of cellobiose.

In a further preferred embodiment of the invention, cellobiose esters according to formula I are prepared 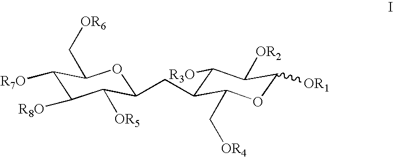
in which the radicals R₁ to R₈, in each case independently of one another, are H or an acyl radical, and the groups of the acyl radicals bonded to the CO group are chosen from linear or branched saturated and unsaturated hydrocarbon groups having 1 to 29, preferably 7 to 21, carbon atoms or from cyclic or aromatic hydrocarbon groups having 4 to 29 carbon atoms, with the proviso that the number of acyl groups is greater than 0.

In a further preferred embodiment of the invention, oligo- or polysaccharides according to formula II are prepared 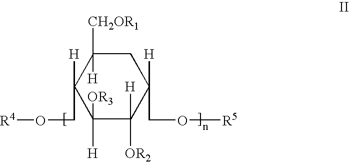
in which the radicals R₁ to R₅, in each case independently of one another, are H or an acyl radical, and the groups of the acyl radicals bonded to the CO group are chosen from linear or branched saturated and unsaturated hydrocarbon groups having 1 to 29, preferably 7 to 21, carbon atoms or from cyclic or aromatic hydrocarbon groups having 4 to 29 carbon atoms, and the degree of polymerization n is preferably 3 to 200, particularly preferably 5 to 100 and extraordinarily preferably 10 to 50, with the proviso that the number of acyl groups is greater than 0.

In a particularly preferred embodiment of the invention, activated carboxylic acids whose basis acid is palmitic acid are used in the process according to the invention. Extraordinary preference is given to preparing substances chosen from dextrin palmitate and cellobiose palmitate by the process according to the invention. In this connection, the dextrins preferably have a degree of polymerization from 3 to 200, particularly preferably from 5 to 100 and especially preferably from 10 to 50.

The degree of esterification per sugar unit is, in the case of disaccharides, preferably from 0.1 to 4 and particularly preferably from 2 to 4.

In the case of oligo- and polysaccharides with degrees of polymerization of from 3 to 200, the degree of esterification per sugar unit is preferably at least 0.1, i.e. from 0.1 to 3.67 for saccharides where n=3, from 0.1 to 3.5 for saccharides where n=4, etc. For oligo- and polysaccharides with degrees of polymerization of from 3 to 200, the degree of esterification per sugar unit is particularly preferably from 0.1 to 3 and extremely preferably from 2 to 3.

In the process according to the invention, the molar ratio of activated carboxylic acid to sugar unit is, in the case of the use of disaccharides, preferably from 0.1:1 to 4:1 and particularly preferably from 2:1 to 4:1.

In the process according to the invention, the molar ratio of activated carboxylic acid to sugar unit is, in the case of the use of oligo- and polysaccharides with degrees of polymerization of from 3 to 200, preferably at least 0.1:1. In the process according to the invention, the molar ratio of activated carboxylic acid to sugar unit, in the case of the use of oligo- and polysaccharides with degrees of polymerization of from 3 to 200, particularly preferably from 0.1:1 to 3:1 and extraordinarily preferably from 2:1 to 3:1.

In a further preferred embodiment of the invention, cellobiose octanonoate is prepared.

The process according to the invention is preferably carried out at a temperature from 45 to 80° C., particularly preferably at a temperature from 65 to 75° C.

In a further preferred embodiment, the process is carried out in such a way that the solvent or solvent mixture is recycled.

The examples below are intended to illustrate the subject matter of the invention in more detail, but not limit it thereto.

Water can be removed from the sugar component either with or without entrainer (petroleum ether), as shown in the examples. Also, for the neutralization of the reaction mixture, it is possible to use either an aqueous base, or an alcoholic base. This will be demonstrated in the examples.

### EXAMPLE 1

### Preparation of Cellobiose Palmitate

A stirrable flask fitted with vacuum adapter is initially charged with 12.5 g of hydrous cellobiose and 120 g of 2-methylpyrrolidone (NMP), and 20% of the NMP used is distilled off again at a maximum of 110° C. and 50 mbar. The mixture is then cooled to 70° C. and, over the course of 60 minutes at 70-75° C., 1 mol of palmitoyl chloride per hydroxyl group of the cellobiose is metered in. After a post-reaction of 4 hours at 70-75° C., the mixture is added dropwise, with the simultaneous metered addition of 23 g of NaOH (50% strength by weight), into an initial charge of 200 g of isopropanol (duration: 45-60 minutes). The pH is between 2 and 4. When this precipitation reaction is complete, this isopropanolic mixture is adjusted to pH 10 (10% tq aqueous) using NaOH (50% strength by weight). The precipitate is then filtered off with suction and washed with demineralized water (adjusted to pH 10) and pure demineralized water, in each case for 30 minutes at 40-45° C. The washed precipitate is dried overnight at 50° C. under reduced pressure (20-50 mbar). The yield of finely crystalline product is 70% (melting point about 120° C.).

### EXAMPLE 2

### Preparation of Dextrin Palmitate

A stirrable flask fitted with water separator is charged with hydrous dextrin (17.8 g) and petroleum ether (boiling range 70-90° C.), and water is removed azeotropically for 4 hours at 74-78° C. Then, approximately 75% of the petroleum ether used is distilled off again. 100 g of 2-methylpyrrolidone (NMP) are added, and the remaining petroleum ether is distilled off up to a still temperature of 130° C. The mixture is then cooled to 70° C. and, over the course of 60 minutes at 70-75° C., palmitoyl chloride (82.5 g) is metered in. After a post-reaction time of 4 hours at 70-75° C., the mixture is then metered into 150 g of isopropanol over the course of 45-60 minutes, with the simultaneous metered addition of isopropanolic KOH (15% strength by weight) at pH 2-4. The precipitate which forms is then adjusted to pH 10 (10% tq aqueous) with isopropanolic KOH and filtered off with suction. The precipitate is washed with demineralized water (adjusted to pH 10) and pure demineralized water for 30 minutes at 40-45° C. The washed precipitate is then dried overnight at 50° C. and 20-50 mbar. The yield of dextrin palmitate is 79.5%.

### COMPARATIVE EXAMPLE

### Preparation of Dextrin Palmitate in the Presence of Pyridine

16.2 g of dried dextrin and 94.9 g of anhydrous pyridine are introduced into a 500 ml stirrable flask. Then, with stirring, 82.5 g of palmitoyl chloride are added dropwise. Slight warming to 40-50° C. occurs. The mixture is stirred at 60° C. for 4 hours. The mixture is then poured into 350 g of water and adjusted to pH 1.6-2.0 with 85 g of conc. hydrochloric acid. The mixture is then after-stirred for a few minutes and then filtered with suction, and the moist product is dissolved in 300 g of petroleum ether (boiling range 60-70° C.). The mixture is then admixed with 300 g of 1% strength hydrochloric acid and stirred at 40° C. for 0.5 hours. This mixture is then transferred to a 2 liter separating funnel, the organic phase is separated from the aqueous phase and the organic phase is evaporated to dryness on a rotary evaporator under slightly reduced pressure at 30-40° C. The solid residue is then taken up in 300 g of ethyl acetate (anhydrous) and recrystallized. The recrystallization is repeated where necessary.

Comparison of the products and yields from the pyridine variant and the 2-methylpyrrolidone (NMP) variant:

| | Pyridine variant (comparative | NMP variant |
|---|---|---|
| | example) | (example 2) |
| Appearance at room | Pale yellow powder | White, powdery to |
| temperature | | granular |
| Yield | 37.0% | 79.5% |
| Acid number | 3.4 mg of KOH/g | 0.92 mg of KOH/g |
| Chloride content | Not determined | 0.10% |
| Melting point | ca. 99° C. | ca. 97° C. |

1. a process for the preparation of a saccharide ester comprising the step of reacting a saccharide with an activated carboxylic acid in a dipolar-aprotic solvent and in the absence of a basic amine compound:

2. The process as claimed in claim 1, wherein the dipolar-aprotic solvent is a lactam.

3. The process as claimed in claim 2, wherein the lactam is 2-methylpyrrolidone.

4. The process as claimed in claim 1, wherein the saccharide is dextrin or cellobiose.

5. The process as claimed in claim 1, wherein the activated carboxylic acid is selected from the group consisting of carbonyl chlorides and carboxylic anhydrides.

6. The process as claimed in claim 1, wherein the acid on which the activated carboxylic acid is based is palmitic acid.

7. The process as claimed in claim 1, wherein the saccharide is a disaccharide and wherein the molar ratio of activated carboxylic acid to sugar unit in the disaccharide is from 0.1:1 to 4:1.

8. The process as claimed in claim 1, wherein the saccharide is an an oligo- or polysaccharide with a degree of polymerization of from 3 to 200 and wherein the molar ratio of activated carboxylic acid to sugar unit in the oligo- or polysaccharide is at least 0.1:1.

9. The process as claimed in claim 1, carried out at a temperature of from 45 to 80° C.

10. The process as claimed in claim 1, wherein the solvent is recycled.

11. Cellobiose octanonanoate prepared by the process of claim 1.

12. A saccharide ester made by the process of claim 1.
